(19) United States
(12) Patent Application Publication
(10) Pub. No.: US 2009/0232776 A1
(43) Pub. Date: Sep. 17, 2009

(54) USE OF IMMUNOMODULATORY COMPOUNDS FOR THE TREATMENT OF DISORDERS ASSOCIATED WITH ENDOTHELIAL DYSFUNCTION

(76) Inventor: Laure A. Moutouh-de Parseval, Summit, NJ (US)

Correspondence Address: JONES DAY, 222 EAST 41ST ST, NEW YORK, NY, 10017, US

(21) Appl. No.: 12/267,426

(22) Filed: Nov. 7, 2008

Related U.S. Application Data
(60) Provisional application No. 61/002,411, filed on Nov. 8, 2007.

(51) Int. Cl. A61K 35/12; A61K 31/454; A61P 17/02

(52) U.S. Cl. 424/93.7; 514/323

(57) ABSTRACT

Methods of treating, preventing or managing endothelial dysfunction and other disorders are disclosed. The methods encompass the administration of an immunomodulatory compound provided herein. Further described are methods of treatment using the immunomodulatory compounds in combination with a second active agent. Pharmaceutical compositions and single unit dosage forms suitable for use in the methods provided herein are also disclosed.

This application claims priority to U.S. Provisional Application No. 61/002,411, filed Nov. 8, 2007, which is incorporated herein by reference in its entirety.

### 1. FIELD

Provided herein are methods of treating, preventing or managing various disorders associated with endothelial dysfunction. Pharmaceutical compositions and dosing regimens using the immunomodulatory compounds, optionally with other second active agents, are also provided.

### 2. BACKGROUND

### 2.1 Endothelial Dysfunction

Endothelial dysfunction is a physiological dysfunction of normal biochemical processes carried out by the endothelium, the cells that line the inner surface of blood vessels, e.g., arteries and veins. Endothelial dysfunction is characterized by compromise of normal function of endothelial cells, which includes mediation of coagulation, platelet adhesion, immune function, control of volume and electrolyte content of intravascular and extravascular spaces.

Although endothelial dysfunction can result from a variety of factors, one of the factors is the impairment of differentiation of endothelial progenitor cells (“EPCs”) into functional endothelial cells. Generally, two approaches of therapeutics are currently employed to increase the EPCs, and thus, enhance the differentiation into functional endothelial cells. The first approach is directed to the expansion and mobilization of EPCs using the growth factors such as GM-CSF, G-CSF, and VEGF, or small molecules such as statins. The second approach is directed to the transplantation of EPCs using intravenous infusion into the general circulation or intra aortic infusion at the site of ischemia.

Although those approaches may be practiced with varying degree of success, a need exists for an effective method of enhancing the differentiation of EPCs into normal and functional endothelial cells.

### 2.2 Immunomodulatory Drugs

A number of studies have been conducted with the aim of providing compounds that can safely and effectively be used to treat diseases associated with abnormal production of TNF-α. See, e.g., Marriott, J. B., et al., Expert Opin. Biol. Ther. 1(4):1-8 (2001); G. W. Muller, et al., Journal of Medicinal Chemistry 39(17): 3238-3240 (1996); and G. W. Muller, et al., Bioorganic & Medicinal Chemistry Letters 8: 2669-2674 (1998). Some studies have focused on a group of compounds selected for their capacity to potently inhibit TNF-α production by LPS stimulated PBMC. L. G. Corral, et al., Ann. Rheum. Dis. 58:(Suppl I) 1107-1113 (1999). These compounds, which are referred to as—Immunomodulatory Drugs, show not only potent inhibition of TNF-α but also marked inhibition of LPS induced monocyte IL1β and IL12 production. LPS induced IL6 is also inhibited by immunomodulatory compounds, albeit partially. These compounds are potent stimulators of LPS induced IL10. Id. Particular examples of Immunomodulatory Drugs include, but are not limited to, the substituted 2-(2,6-dioxopiperidin-3-yl) phthalimides and substituted 2-(2,6-dioxopiperidin-3-yl)-1-oxoisoindoles described in U.S. Pat. Nos. 6,281,230 and 6,316,471, both to G. W. Muller, et al.

### 3. SUMMARY

Provided herein are methods of enhancing the differentiation of EPCs into functional endothelial cells. In one embodiment, the methods comprise contacting an immunomodulatory compound provided herein, or a pharmaceutically acceptable salt, solvate (e.g., hydrate), stereoisomer, or prodrug thereof, with an EPC, optionally in the presence of growth factors such as, but not limited to, VEGF, FGF, and SCF. In another embodiment, the methods comprise administering to a patient suffering from, or likely to suffer from, endothelial dysfunction an effective amount of an immunomodulatory compound provided herein, or a pharmaceutically acceptable salt, solvate (e.g., hydrate), stereoisomer, or prodrug thereof, optionally in combination with growth factors such as, but not limited to, VEGF, FGF, and SCF.

Also provided herein are methods of treating and preventing various disorders associated with endothelial dysfunction. The methods comprise administering to a patient a therapeutically or prophylactically effective amount of an immunomodulatory compound, or a pharmaceutically acceptable salt, solvate (e.g., hydrate), stereoisomer, or prodrug thereof.

Also provided herein are methods of managing various disorders associated with endothelial dysfunction, which comprise administering to a patient a therapeutically or prophylactically effective amount of an immunomodulatory compound provided herein, or a pharmaceutically acceptable salt, solvate, stereoisomer, or prodrug thereof.

In some embodiments, an immunomodulatory compound is administered in combination with a therapy conventionally used to treat, prevent or manage the disorders associated with endothelial dysfunction. Examples of such conventional therapies include, but are not limited to, chemical agents and adaptive immunotherapy.

Also provided herein are pharmaceutical compositions, single unit dosage forms, dosing regimens and kits which comprise an immunomodulatory compound, or a pharmaceutically acceptable salt, solvate, stereoisomer, or prodrug thereof, and a second, or additional, active agent. Second active agents include specific combinations, or “cocktails,” of drugs.

### 4. BRIEF DESCRIPTION OF FIGURES

FIG. 1 is a schematic illustration of procedures for investigating the effect of an immunomodulatory compound on endothelial differentiation of CD133⁺ progenitor cells.

FIG. 2 illustrates the morphology of CD133⁺ cells differentiated with endothelial growth factor in the presence of an immunomodulatory compound 2-amino-N-(2-(3-methyl-2,6-dioxopiperidin-3-yl)-1,3-dioxoisoindolin-4-yl)acetamide hydrochloride.

FIG. 3A is a summary table of the phenotype of CD133⁺ cells differentiated in the presence of growth factor and immunomodulatory compound 2-amino-N-(2-(3-methyl-2,6-dioxopiperidin-3-yl)-1,3-dioxoisoindolin-4-yl)acetamide hydrochloride or of growth factor only, with different serums (FBS and HS: condition 1; or FBS only: condition 2).

FIG. 3B illustrates surface expression of VEGFR-2 (KDR) in various conditions denoted.

FIG. 3C illustrates surface expression of VE-cadherin in various conditions denoted.

FIG. 3D is a summary table of CD133+ cells differentiated in the presence of various factors denoted.

FIG. 4 illustrates functional characterization of CD133+ cells differentiated toward the endothelial lineage. Acetyl LDL reuptake by UAE1-lectin expressing cells, cultured in the presence of growth factor only, 0.05% and 0.1% DMSO, 2-amino-N-(2-(3-methyl-2,6-dioxopiperidin-3-yl)-1,3-dioxoisoindolin-4-yl)acetamide hydrochloride, 1-oxo-2-(2,6-dioxopiperidin-3-yl)-4-aminoisoindoline, and thalidomide, was measured using flow cytometry.

FIG. 5 illustrates the modulation of endothelial markers in CD133+ cells cultured in the presence of growth factor only, 2-amino-N-(2-(3-methyl-2,6-dioxopiperidin-3-yl)-1,3-dioxoisoindolin-4-yl)acetamide hydrochloride, DMSO, 1,3-dioxo-2-(2,6-dioxopiperidin-3-yl)-4-aminoisoindoline, 1-oxo-2-(2,6-dioxopiperidin-3-yl)-4-aminoisoindoline, and thalidomide.

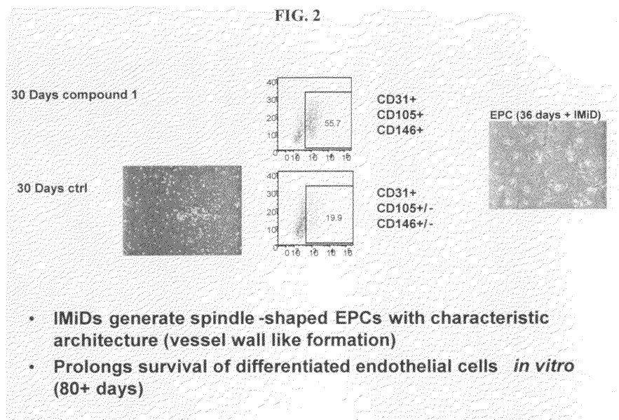

In FIGS. 2, 3A, 3B, 3C, 3D, 4 and 5, Compound 1 refers to 2-amino-N-(2-(3-methyl-2,6-dioxopiperidin-3-yl)-1,3-dioxoisoindolin-4-yl)acetamide hydrochloride.

### 5. DETAILED DESCRIPTION

In one embodiment, provided are methods of enhancing the differentiation of EPCs into functional endothelial cells. In one embodiment, the methods comprise contacting an immunomodulatory compound provided herein, or a pharmaceutically acceptable salt, solvate (e.g., hydrate), stereoisomer, or prodrug thereof, with an EPC, optionally in the presence of growth factors such as, but not limited to, VEGF, FGF, and SCF. In another embodiment, the methods comprise administering to a patient suffering from, or likely to suffer from, endothelial dysfunction an effective amount of an immunomodulatory compound provided herein, or a pharmaceutically acceptable salt, solvate (e.g., hydrate), stereoisomer, or prodrug thereof, optionally in combination with growth factors such as, but not limited to, VEGF, FGF, and SCF.

In another embodiment, provided are methods of treating, managing, and/or preventing disorders associated with endothelial dysfunction, which comprise administering to a patient a therapeutically or prophylactically effective amount of an immunomodulatory compound provided herein, or a pharmaceutically acceptable salt, solvate, stereoisomer, or prodrug thereof.

In some embodiments, the immunomodulatory compound is administered in combination with another drug (“second active agent”) or method of treating, managing, and/or preventing such disorders. Second active agents include, but are not limited to, small molecules and large molecules (e.g., proteins and antibodies), examples of which are provided herein.

Also provided are pharmaceutical compositions (e.g., single unit dosage forms) that can be used in methods disclosed herein. Particular pharmaceutical compositions comprise an immunomodulatory compound provided herein, or a pharmaceutically acceptable salt, solvate, stereoisomer, or prodrug thereof, and a second active agent.

### 5.1 DEFINITIONS

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases known in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of immunomodulatory compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of immunomodulatory compounds that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elsevier, New York 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyl-oxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

The immunomodulatory compound for use in the methods and compositions contains a chiral center, and thus can exist as a racemic mixture of R and S enantiomers. The methods and compositions provided herein encompass the use of stereomerically pure forms of this compound, as well as the use of mixtures of those forms. For example, mixtures comprising equal or unequal amounts of the enantiomers may be used in methods and compositions provided herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

As used herein and unless otherwise indicated, the term “stereomerically pure” means that a compound substantially comprises one stereoisomer, and is substantially free of other stereoisomers. For example, a stereomerically pure compound having one chiral center will substantially comprise one enantiomer and will be substantially free of the opposite enantiomer. A stereomerically pure compound having two chiral centers will substantially comprise one stereoisomer (e.g., diastereoisomer) and will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. As used herein and unless otherwise indicated, the term “stereomerically enriched” means a composition that comprises greater than about 55% by weight of one stercoisomer of a compound, 60% by weight of one stereoisomer of a compound, greater than about 70% by weight, or greater than about 75% by weight of one stereoisomer of a compound. As used herein and unless otherwise indicated, the term “enantiomerically pure” means a stereomerically pure composition of a compound having one chiral center. Similarly, the term “stereomerically enriched” means a stereomerically enriched composition of a compound having one chiral center. In other words, the methods provided herein encompass the use of the R or S enantiomer of the immunomodulatory compound.

As used herein, unless otherwise specified, the term “treating” refers to the administration of a compound provided herein, with or without other additional active agent, after the onset of symptoms of the particular disease.

As used herein, unless otherwise specified, the term “preventing” refers to the treatment with or administration of an immunomodulatory compound, with or without other additional active compound, prior to the onset of symptoms, particularly to patients at risk of endothelial dysfunction and/or disorders associated therewith. The term “prevention” includes the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

As used herein and unless otherwise indicated, the term “managing” encompasses treating a patient who had suffered from the particular disease in an attempt to prevent or minimize the recurrence of the disease and/or reducing mortality rates of the patients.

### 5.2 IMMUNOMODULATORY COMPOUNDS

As used herein and unless otherwise indicated, the terms “immunomodulatory compounds” encompass certain small organic molecules that inhibit LPS induced monocyte TNF-α, IL-1β, IL-12, IL-6, MIP-1α, MCP-1, GM-CSF, G-CSF, and COX-2 production. Specific immunomodulatory compounds are discussed below.

TNF-α is an inflammatory cytokine produced by macrophages and monocytes during acute inflammation. TNF-α is responsible for a diverse range of signaling events within cells. Without being limited by a particular theory, one of the biological effects exerted by the immunomodulatory compounds provided herein is the reduction of myeloid cell TNF-α production. Immunomodulatory compounds of provided herein may enhance the degradation of TNF-α mRNA.

Specific examples of immunomodulatory compounds include cyano and carboxy derivatives of substituted styrenes such as those disclosed in U.S. Pat. No. 5,929,117; 1-oxo-2-(2,6-dioxo-3-fluoropiperidin-3-yl) isoindolines and 1,3-dioxo-2-(2,6-dioxo-3-fluoropiperidine-3-yl) isoindolines such as those described in U.S. Pat. Nos. 5,874,448 and 5,955,476; the tetra substituted 2-(2,6-dioxopiperidin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide), substituted 2-(2,6-dioxopiperidin-3-yl) phthalimides and substituted 2-(2,6-dioxopiperidin-3-yl)-1-oxoisoindoles including, but not limited to, those disclosed in U.S. Pat. Nos. 5,635,517, 6,281,230, 6,316,471, 6,403,613, 6,476,052 and 6,555,554; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; and isoindole-imide compounds such as those described in U.S. patent publication no. 2003/0045552 published on Mar. 6, 2003, U.S. patent publication no. 2003/0096841 published on May 22, 2003, and International Application No. PCT/US01/50401 (International Publication No. WO 02/059106). The entireties of each of the patents and patent applications identified herein are incorporated herein by reference. Immunomodulatory compounds do not include thalidomide.

Various immunomodulatory compounds provided herein contain one or more chiral centers, and can exist as racemic mixtures of enantiomers or mixtures of diastereomers. The methods and compositions herein encompass the use of stereomerically pure forms of such compounds, as well as the use of mixtures of those forms. For example, mixtures comprising equal or unequal amounts of the enantiomers of a particular immunomodulatory compounds may be used in methods and compositions provided herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

In one embodiment, immunomodulatory compounds provided include, but are not limited to, 1-oxo- and 1,3 dioxo-2-(2,6-dioxopiperidin-3-yl) isoindolines substituted with amino in the benzo ring as described in U.S. Pat. No. 5,635,517 which is incorporated herein by reference. These compounds have the structure I:

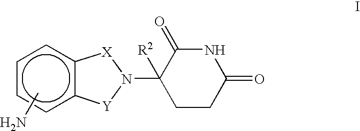

in which one of X and Y is C═O, the other of X and Y is C═O or CH₂, and R² is hydrogen or lower alkyl, in particular methyl. Specific immunomodulatory compounds include, but are not limited to:

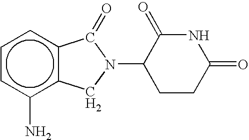

1-oxo-2-(2,6-dioxopiperidin-3-yl)-4-aminoisoindoline;

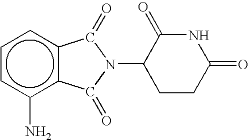

1,3-dioxo-2-(2,6-dioxopiperidin-3-yl)-4-aminoisoindoline; and

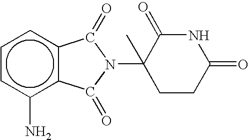

1,3-dioxo-2-(3-methyl-2,6-dioxopiperidin-3-yl)-4-aminoisoindole, and optically pure isomers thereof. The compounds can be obtained via standard, synthetic methods (see e.g., U.S. Pat. No. 5,635,517, incorporated herein by reference). The compounds are also available from Celgene Corporation, Warren, N.J.

Other specific immunomodulatory compounds belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference. Representative compounds are of formula:

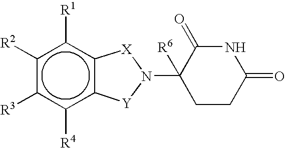

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzyl, or halo;

provided that R⁶ is other than hydrogen if X and Y are C═O and (i) each of R¹, R², R³, and R⁴ is fluoro or (ii) one of R¹, R², R³, or R⁴ is amino.

Compounds representative of this class are of the formulas:

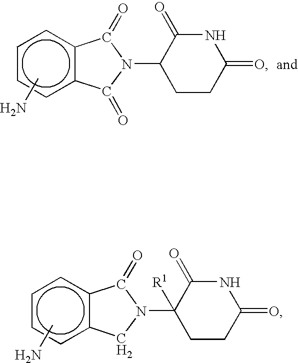

wherein R¹ is hydrogen or methyl. In a separate embodiment, the methods and compositions provided herein encompass the use of enantiomerically pure forms (e.g. optically pure (R) or (S) enantiomers) of these compounds.

Still other specific immunomodulatory compounds belong to a class of isoindole-imides disclosed in U.S. Patent Application Publication Nos. US 2003/0096841 and US 2003/0045552, and International Application No. PCT/US01/50401 (International Publication No. WO 02/059106), each of which are incorporated herein by reference. Representative compounds are of formula II:

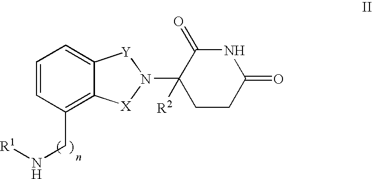

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl)-C₁-C₆)heterocycloalkyl, (C₀-C₄)alkyl)-C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-C₁-C₆)heterocycloalkyl, (C₀-C₄)alkyl)-C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl R⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;

n is 0 or 1; and

* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and

R³ is (C₁-C₈)alkyl, (C₃-C₇)cycloalkyl, (C₂-C₈)alkenyl, (C₂-C₈)alkynyl, benzyl, aryl, (C₀-C₄)alkyl C₁-C₆)heterocycloalkyl, (C₀-C₄)alkyl(C₂-C₅)heteroaryl, (C₅-C₈)alkyl-N(R⁶)₂; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

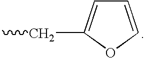

In another embodiment of the compounds of formula II, R¹ is

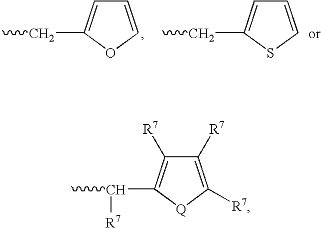

wherein Q is O or S, and each occurrence of R⁷ is independently H,(C₁-C₈)alkyl, (C₃-C₇)cycloalkyl, (C₂-C₈)alkenyl, (C₂-C₈)alkynyl, benzyl, aryl, halogen, (C₀-C₄)alkyl)-C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C₀-C₄)alkyl-(C₂-C₅)heteroaryl, (C₁-C₈)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl}methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; and N-{[2-(2,6-dioxo(3-piperidyl))-1,3-dioxoisoindolin-4-yl]methyl}(benzylamino)carboxamide.

Still other specific immunomodulatory compounds belong to a class of isoindole-imides disclosed in U.S. Patent Application Publication Nos. US 2002/0045643, International Publication No. WO 98/54170, and U.S. Pat. No. 6,395,754, each of which is incorporated herein by reference. Representative compounds are of formula III:

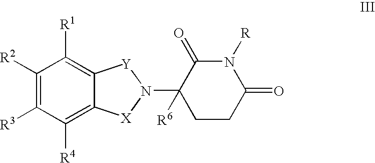

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

* represents a chiral-carbon center.

Other representative compounds are of formula:

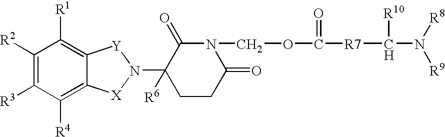

wherein:

one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—; and

R¹⁰ is hydrogen, alkyl of to 8 carbon atoms, or phenyl.

Other representative compounds are of formula:

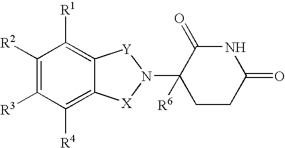

in which

one of X and Y is C═O and the other of X and Y is C═O or CH₂;

each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and

R⁶ is hydrogen, alkyl of 1 to 8 carbon atoms, benzo, chloro, or fluoro.

Other representative compounds are of formula:

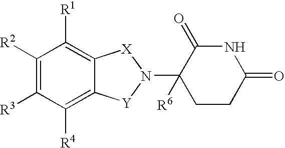

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and

R⁶ is alkyl of 1 to 8 carbon atoms, benzo, chloro, or fluoro.

Specific examples of the compounds are of formula:

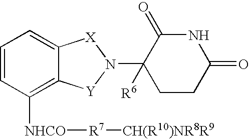

in which:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or —(CₙH₂n)— in which n has a value of 0 to 4; each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl.

Other specific immunomodulatory compounds include, but are not limited to, 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476, each of which is incorporated herein by reference. Representative compounds are of formula:

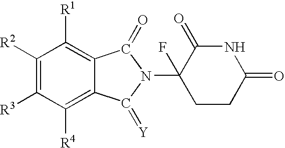

wherein:

Y is oxygen or H₂ and

each of R¹, R², R³, and R⁴, independently of the others, is hydrogen, halo, alkyl of 1 to 4 carbon atoms, alkoxy of 1 to 4 carbon atoms, or amino.

Other specific immunomodulatory compounds include, but are not limited to, the tetra substituted 2-(2,6-dioxopiperidin-3-yl)-1-oxoisoindolines described in U.S. Pat. No. 5,798,368, which is incorporated herein by reference. Representative compounds are of formula:

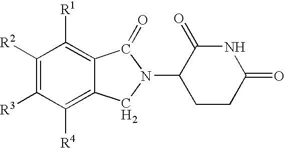

wherein each of R¹, R², R³, and R⁴, independently of the others, is halo, alkyl of 1 to 4 carbon atoms, or alkoxy of 1 to 4 carbon atoms.

Other specific immunomodulatory compounds include, but are not limited to, 1-oxo and 1,3-dioxo-2-(2,6-dioxopiperidin-3-yl) isoindolines disclosed in U.S. Pat. No. 6,403,613, which is incorporated herein by reference. Representative compounds are of formula:

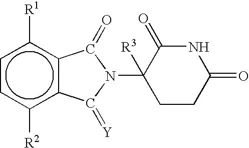

in which

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula:

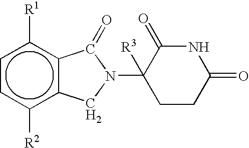

wherein

a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and

R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other representative compounds are of formula:

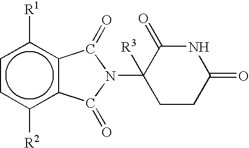

wherein:

a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and

R³ is hydrogen, alkyl of from 1 to 4 carbon atoms, or benzyl.

Other specific immunomodulatory compounds include, but are not limited to, 1-oxo and 1,3-dioxoisoindolines substituted in the 4- or 5-position of the indoline ring described in U.S. Pat. No. 6,380,239 and U.S. application publication no. 2006/0084815, published Apr. 20, 2006, which are incorporated herein by reference. Representative compounds are of formula:

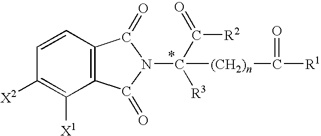

in which the carbon atom designated C* constitutes a center of chirality (when n is not zero and R¹ is not the same as R²); one of X¹ and X² is amino, nitro, alkyl of one to six carbons, or NH-Z, and the other of X¹ or X² is hydrogen; each of R¹ and R² independent of the other, is hydroxy or NH-Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further representative compounds are of formula:

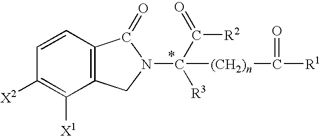

in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl or an alkyl or acyl of one to six carbons; and n has a value of 0, 1, or 2.

Specific examples include, but are not limited to, 2-(4-amino-1-oxo-1,3-dihydro-isoindol-2-yl)-4-carbamoyl-butyric acid and 4-(4-amino-1-oxo-1,3-dihydro-isoindol-2-yl)-4-carbamoyl-butyric acid, which have the following structures, respectively, and pharmaceutically acceptable salts, solvates, prodrugs, and stereoisomers thereof:

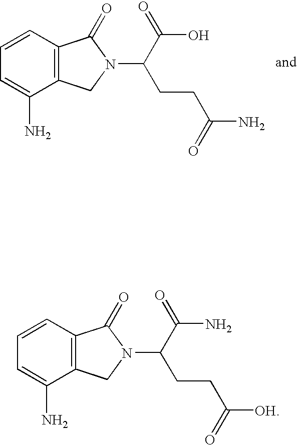

Other representative compounds are of formula:

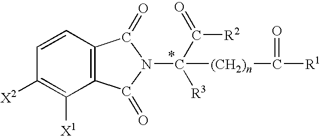

in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, and 2-{4-[(furan-2-yl-methyl)-amino]-1,3-dioxo-1,3-dihydro-isoindol-2-yl}-pentanedioic acid, which have the following structures, respectively, and pharmaceutically acceptable salts, solvate, prodrugs, and stereoisomers thereof:

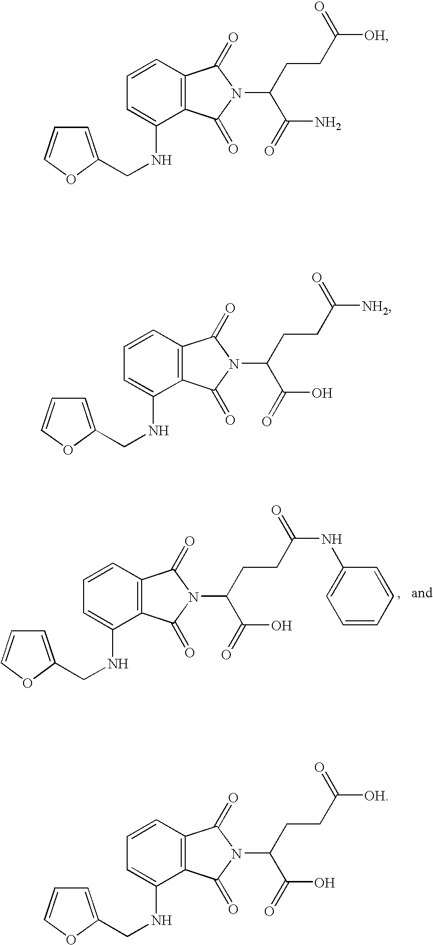

Other specific examples of the compounds are of formula:

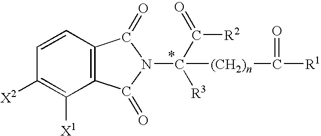

wherein:

one of X¹ and X² is nitro, or NH-Z, and the other of X¹ or X² is hydrogen;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Other representative compounds are of formula:

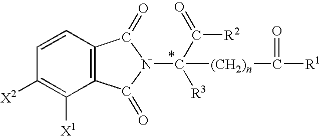

wherein:

one of X¹ and X² is alkyl of one to six carbons;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Still other specific immunomodulatory compounds include, but are not limited to, isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula:

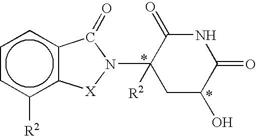

wherein:

the carbon atoms designated * constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen; and

R³ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms,

benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms, or

benzyl, unsubstituted or substituted with alkyl of 1 to 8 carbon atoms, alkoxy of 1 to 8 carbon atoms, halo, amino, or alkylamino of 1 to 4 carbon atoms.

All of the compounds described can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques.

Compounds used herein may be small organic molecules having a molecular weight less than about 1,000 g/mol, and are not proteins, peptides, oligonucleotides, oligosaccharides or other macromolecules.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 5.3 METHODS OF TREATMENT, PREVENTION AND MANAGEMENT

Provided herein are methods of treating, preventing and/or managing endothelial dysfunction and/or various disorders associated with endothelial dysfunction. Without limited by a particular theory, mobilization of EPCs is a part of a normal physiological response in the process of repair of cellular and tissue damages. Certain physiological events and response factors drive EPCs to the site of neovascularization and allow EPCs to differentiate into endothelial cells in vivo. Further without limited by a particular theory, it is believed that immunomodulatory compounds provided herein can enhance these natural processes of differentiation in an expeditious and controllable manner.

Methods provided herein comprise administering one or more immunomodulatory compound provided herein, or a pharmaceutically acceptable salt, solvate, stereoisomer, or prodrug thereof, to a patient (e.g., a human) suffering, or likely to suffer, from endothelial dysfunction and/or various disorders associated with endothelial dysfunction, e.g., those provided herein.

In one embodiment of the invention, an immunomodulatory compound provided herein can be administered orally and in single or divided daily doses in an amount of from about 0.10 to about 150 mg/day. In a particular embodiment, 4-(amino)-2-(2,6-dioxo(3-piperidyl))-isoindoline-1,3-dione may be administered in an amount of from about 0.1 to about 1 mg per day, or alternatively from about 0.1 to about 5 mg every other day. In another particular embodiment, 3-(4-amino-1-oxo-1,3-dihydro-isoindol-2-yl)-piperidine-2,6-dione may be administered in an amount of from about 1 to about 25 mg per day, or alternatively from about 10 to about 50 mg every other day. In another particular embodiment, 2-amino-N-(2-(3-methyl-2,6-dioxopiperidin-3-yl)-1,3-dioxoisoindolin-4-yl)acetamide hydrochloride may be administered in an amount of from about 0.10 to about 150 mg/day.

In one embodiment, 4-(amino)-2-(2,6-dioxo(3-piperidyl))-isoindoline-1,3-dione may be administered in an amount of about 1, 2, or 5 mg per day to patients. In a particular embodiment, 3-(4-amino-1-oxo-1,3-dihydro-isoindol-2-yl)-piperidine-2,6-dione may be administered initially in an amount of 1 mg/day and the dose can be escalated every week to 10, 20, 25, 30 and 50 mg/day. In another embodiment, 3-(4-amino-1-oxo-1,3-dihydro-isoindol-2-yl)-piperidine-2,6-dione can be administered in an amount of up to about 30 mg/day to patients. In another embodiment, 3-(4-amino-1-oxo-1,3-dihydro-isoindol-2-yl)-piperidine-2,6-dione can be administered in an amount of up to about 40 mg/day to patients.

In one embodiment, 4-(amino)-2-(2,6-dioxo(3-piperidyl))-isoindoline-1,3-dione may be administered in an amount of from about 0.1 to about 1 mg per day, or alternatively from about 0.1 to about 5 mg every other day, to patients.

In another embodiment, 3-(4-amino-1-oxo-1,3-dihydro-isoindol-2-yl)-piperidine-2,6-dione may be administered in an amount of from about 1 to about 25 mg per day, or alternatively from about 10 to about 50 mg every other day, to patients.

Also provided herein are methods of treating, preventing, and/or managing endothelial dysfunction and/or various disorders associated therewith using an ex vivo therapy. The methods comprise contacting EPCs with an immunomodulatory compound and administering to a patient (e.g., inoculating) the EPCs which have been contacted with the immunomodulatory compound. Without limited by a particular theory, it is believed that immunomodulatory compounds provided herein may stimulate or enhance the expansion of EPCs ex vivo, and the EPCs so expanded may be administered to a patient for the treatment, prevention, and/or management of endothelial dysfunction and/or various disorders associated therewith, e.g., those provided herein.

Examples of disorders treated, prevented or managed by immunomodulatory compounds provided herein include, but are not limited to: cardiovascular disorders such as, but not limited to, arterial hypertension, orthostatic hypotension, syncope, arteriosclerosis, coronary artery disease, heart failure, shock, arrhythmias, valvular heart disease, endocarditis, pericardial disease, peripheral artery disease, cardiac tumors, and diseases of the aorta and branches thereof; diabetes; systemic sclerosis and chronic renal failure.

Examples of arterial hypertension include, but are not limited to, renovascular hypertension and hypertensive encephalopathy. In one embodiment, the disorder to be treated, prevented, and/or managed using the methods provided herein is arterial hypertension.

Examples of arteriosclerosis include, but are not limited to, atherosclerosis and nonatheromatous arteriosclerosis. In one embodiment, the disorder to be treated, prevented, and/or managed using the methods provided herein is arteriosclerosis. In one embodiment, the disorder to be treated, prevented, and/or managed using the methods provided herein is nonatheromatous arteriosclerosis. In one embodiment, the disorder to be treated, prevented, and/or managed using the methods provided herein is not atherosclerosis.

Examples of heart failures include, but are not limited to, congestive heart failure, cardiomyopathy, chronic heart failure, and pulmonary hypertension. In one embodiment, the disorder to be treated, prevented, and/or managed using the methods provided herein is heart failure. In one embodiment, the disorder to be treated, prevented, and/or managed using the methods provided herein is chronic heart failure. In one embodiment, the disorder to be treated, prevented, and/or managed using the methods provided herein is not congestive heart failure or pulmonary hypertension.

Examples of valvular heart disease include, but are not limited to, mitral valve disease (e.g., mitral valve prolapse, mitral regurgitation, and mitral stenosis), aortic valve disease (e.g., aortic regurgitation, pulmonary regurgitation, aortic stenosis, and pulmonary stenosis), and tricuspid valve disease (e.g., tricuspid regurgitation and tricuspid stenosis). In one embodiment, the disorder to be treated, prevented, and/or managed using the methods provided herein is a valvular heart disease. In one embodiment, the disorder to be treated, prevented, and/or managed using the methods provided herein is a mitral valve disease. In one embodiment, the disorder to be treated, prevented, and/or managed using the methods provided herein is a aortic valve disease. In one embodiment, the disorder to be treated, prevented, and/or managed using the methods provided herein is a tricuspid valve disease.

Examples of endocarditis include, but are not limited to, infective endocarditis and non-infective endocarditis. In one embodiment, the disorder to be treated, prevented, and/or managed using the methods provided herein is endocarditis.

Examples of disease of aorta and branches thereof include, but are not limited to, aneurysms, aortic dissection, inflammation of the aorta (e.g., Takayasu's arteritis), and occlusion of the aorta and branches thereof. In one embodiment, the disorder to be treated, prevented, and/or managed using the methods provided herein is a disease of aorta and branches thereof.

In one embodiment, the disorder to be treated, prevented, and/or managed using the methods provided herein is orthostatic hypotension. In one embodiment, the disorder to be treated, prevented, and/or managed using the methods provided herein is syncope. In one embodiment, the disorder to be treated, prevented, and/or managed using the methods provided herein is coronary artery disease. In one embodiment, the disorder to be treated, prevented, and/or managed using the methods provided herein is a shock. In one embodiment, the disorder to be treated, prevented, and/or managed using the methods provided herein is arrhythmias. In one embodiment, the disorder to be treated, prevented, and/or managed using the methods provided herein is a pericardial disease (e.g., pericarditis). In one embodiment, the disorder to be treated, prevented, and/or managed using the methods provided herein is peripheral artery disease.

In one embodiment, the disorder to be treated, prevented, and/or managed using the methods provided herein is diabetes. In another embodiment, the disorder to be treated, prevented, and/or managed using the methods provided herein is chronic renal failure. In one embodiment, the disorder to be treated, prevented, and/or managed using the methods provided herein is systemic sclerosis.

In another embodiment, the immunomodulatory compounds provided herein are used to promote wound healing. Without being limited by a particular theory, EPCs have been shown to play a role in wound healing. For example, it was reported that angiogenesis and vasculogenesis may induce the growth of new blood vessels and wound healing by stimulation of bone marroe-derived progenitor cell mobilization and homing. (See Velazquez, Journal of Vascular Surgery, 45(6): A39-47 (2007)). In one embodiment where immunomodulatory compounds are used for promotion of wound healing, the immunomodulatory compound is not 1-oxo-2-(2,6-dioxopiperidin-3-yl)-4-methylisoindoline.

Doses of an immunomodulatory compound disclosed herein, or a pharmaceutically acceptable salt, solvate, stereoisomer or prodrug thereof, vary depending on factors such as: specific indication to be treated, prevented, and/or managed; age and condition of a patient; and amount of second active agent used, if any. Generally, an immunomodulatory compound disclosed herein, or a pharmaceutically acceptable salt, solvate, stereoisomer or prodrug thereof, may be used in an amount of from about 0.1 mg to about 500 mg per day, and can be adjusted in a conventional fashion (e.g., the same amount administered each day of the treatment, prevention or management period), in cycles (e.g., one week on, one week off), or in an amount that increases or decreases over the course of treatment, prevention, or management.

In other embodiments, the dose can be from about 1 mg to about 300 mg, from about 0.1 mg to about 150 mg, from about 1 mg to about 200 mg, from about 10 mg to about 100 mg, from about 0.1 mg to about 50 mg, from about 1 mg to about 50 mg, from about 10 mg to about 50 mg, from about 10 mg to about 25 mg, from about 20 mg to about 30 mg, or from about 1 mg to about 20 mg.

### 5.4 COMBINATION THERAPY WITH A SECOND ACTIVE AGENT

5.4.1 Second Active Agent

An immunomodulatory compound can be used with or combined with other pharmacologically active compounds (“second active agents or ingredients”) in methods and compositions provided herein. It is believed that certain combinations work synergistically in the methods provided herein. Immunomodulatory compounds can also work to alleviate adverse effects associated with certain second active agents, and some second active agents can be used to alleviate adverse effects associated with immunomodulatory compounds of the invention.

One or more second active ingredients or agents can be used in the methods and compositions provided herein together with an immunomodulatory compound. Second active agents can be large molecules (e.g., proteins) or small molecules (e.g., synthetic inorganic, organometallic, or organic molecules).

In one embodiment, where cardiovascular disorder is treated, prevented, and/or managed, the second active agents include, but are not limited to: diuretics such as, but not limited to, hydrochlorothiazide, bendroflumethiazide, chlorothiazide, chlorthalidone, hydroflumethiazide, indapamide, methylclothiazide, metolazone, bumetanide, ethacrynic acid, furosemide, torsemide, amilride, spironolactone, and triamterene; β-blockers such as, but not limited to, acebutolol, betaxolol, propranolol, metoprolol, atenolol, timolol, bisoprolol, carteolol, carvedilol, labetalol, nadolol, penbutolol, and pindolol; Ca channel blockers such as, but not limited to, diltiazem, verapamil, amlodipine, felodipine, isradipine, nicardipine, nifedipine, and nisoldipine; ACE inhibitors such as, but not limited to, benazepril, captopril, enalapril, fosinopril, lisinopril, moexipril, quinapril, ramipril, and trandolapril; an angiotensin receptor blocker such as, but not limited to, irbesartan, losartan, and valsartan; adrenergic blockers such as, but not limited to, clonidine, guanabenz, guanfacine, methyldopa, labetalol, esmolol, phentolamine, doxazosin, prazosin, terazosin, guanadrel, guanethidine, rauwolfia alkaloids, and reserpine; vasodilators such as, but are not limited to, sodium nitro-prusside; nicardipine, fenoldopam, nitroglycerin, enalaprilat, hydlarazine, diazoxide, and minoxidil; antiplatelet agensts such as, but not limited to, aspirin and ticlopidine; Na channel blockers such as, but not limited to, quinidine, procainamide, disopyramide, lidocaine, mexiletine, tocamide, moricizine, phenyloin, flecamide, and propafenone; potassium channel blockers such as, but not limited to, amiodarone, sotalol, and bretylium tosylate; antibiotics such as, but not limited to, penicillin, amoxicillin, ampicillin, clindamycin, cephalexin, cefadroxil, azithromycin, clarithromycin, and cefezolin; and mixtures thereof.

In one embodiment, where diabetes is treated, prevented, and/or managed, the second active agents include, but are not limited to: insulin; sulfonylurea agents such as, but not limited to, tolbutamide, chlorpropamide, acetohexamide, tolazamide, glyburide, and glimepiride; antihyperglycemic agents such as, but are not limited to, metformin, acarbose, troglitazone, and repaglinide; and mixtures thereof.

In one embodiment, an immunomodulatory compound provided herein may be administered in combination with growth factors such as, but not limited to, VEGF, FGF, and SCF.

In some embodiments, an immunomodulatory compound may be combined with other medical procedures for the treatment, prevention or management of endothelial dysfunctions and other disorders provided herein. Examples of such other procedures include, but are not limited to, dietary restrictions, exercise, angioplastry, artery catherization, and surgery.

5.4.2 Combination Therapy

In certain embodiment, the immunomodulatory compound, or a pharmaceutically acceptable salt, solvate (e.g., hydrate), stereoisomer, clathrate, or prodrug thereof, in combination with one or more second active agents such as those disclosed herein and/or in combination with other medical procedures such as those disclosed herein.

Administration of an immunomodulatory compound and the second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. In one embodiment, the immunomodulatory compound provided herein is administered orally. Typical routes of administration for the second active agents or ingredients provided herein are known to those of ordinary skill in the art. See, e.g., Physicians' Desk Reference, (2006).

It is further contemplated that a combination therapy can be used wherein an immunomodulatory compound provided herein is administered in combination with a regimen of known therapy for the disorders provided herein. The combined use of the immunomodulatory compounds provided herein and conventional therapy may provide a unique treatment regimen effective in certain patients. Without being limited by theory, it is believed that immunomodulatory compounds provided herein may provide additive or synergistic effects when given concurrently with other therapy.

In one embodiment of the invention, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated, prevented, or managed, the severity and stage of disease, and the amount(s) of immunomodulatory compounds of the invention, and any optional additional active agents concurrently administered to the patient.

In another embodiment, an immunomodulatory compound is administered in an amount of from about 0.1 mg to about 150 mg/d in combination with a second active agent to patients.

Also provided herein are methods of increasing the dosage of a therapeutic agent safely and effectively administered to a patient, which comprises administering to a patient (e.g., a human) an immunomodulatory compound provided herein, or a pharmaceutically acceptable salt, solvate, stereoisomer, or prodrug thereof. Patients that can benefit by this method are those likely to suffer from an adverse effect associated with a therapeutic agent provided herein. The administration of an immunomodulatory compound provided herein alleviates or reduces adverse effects which are of such severity that it would otherwise limit the amount of the therapeutic agent.

In one embodiment, an immunomodulatory compound of the invention can be administered orally and daily in an amount of from about 0.1 to about 150 mg, from about 1 to about 50 mg, and from about 2 to about 25 mg prior to, during, or after the occurrence of the adverse effect associated with the administration of the therapeutic agent to a patient.

In another embodiment, provided herein are methods of treating, preventing and/or managing disorders provided herein, which comprises administering an immunomodulatory compound of the invention, or a pharmaceutically acceptable salt, solvate, stereoisomer, or prodrug thereof, in conjunction with (e.g. before, during, or after) other medical procedures described herein. The combined use of the immunomodulatory compounds of the invention and the medical procedure(s) may provide a unique treatment regimen that is unexpectedly effective in certain patients. Without being limited by theory, it is believed that immunomodulatory compounds provided herein may provide additive or synergistic effects when given concurrently with such procedures.

For example, immunomodulatory compounds provided herein may be effective in reducing, treating and/or preventing adverse or undesired effects associated with conventional medical procedures used for the treatment of endothelial dysfunction and other disorders provided herein. In one embodiment, one or more immunomodulatory compounds provided herein are administered to a patient prior to, during, or after the occurrence of the adverse effect associated with conventional therapy.

In one embodiment, an immunomodulatory compound of the invention can be administered in an amount of from about 0.1 to about 150 mg, from about 1 to about 25 mg, and from about 2 to about 10 mg daily by oral administration, alone or in combination with a second active agent disclosed herein, prior to, during, or after the use of conventional therapy.

### 5.5 CYCLING THERAPY

In certain embodiments, the prophylactic or therapeutic agents disclosed herein are cyclically administered to a patient. Cycling therapy involves the administration of an active agent for a period of time, followed by a rest for a period of time, and repeating this sequential administration. Cycling therapy can reduce the development of resistance to one or more of the therapies, avoid or reduce the side effects of the therapies, and/or improves the efficacy of the treatment.

Consequently, in one embodiment of the invention, an immunomodulatory compound provided herein is administered daily in a single or divided doses in a four to six week cycle with a rest period of about a week or two weeks. In other embodiments, the frequency, number, and length of dosing cycles may be increased. Thus, also provided herein is the administration of an immunomodulatory compound for more cycles than are typical. In yet another embodiment, an immunomodulatory compound is administered for a greater number of cycles that would typically cause dose-limiting toxicity in a patient to whom a second active ingredient is not also being administered.

In one embodiment, an immunomodulatory compound provided herein is administered daily and continuously for three or four weeks at a dose of from about 0.1 to about 150 mg/d, followed by a break of one or two weeks. 4-(Amino)-2-(2,6-dioxo(3-piperidyl))-isoindoline-1,3-dione is preferably administered daily and continuously at an initial dose of 0.1 to 5 mg/d with dose escalation (every week) by 1 to 10 mg/d to a maximum dose of 50 mg/d for as long as the therapy is tolerated. In a particular embodiment, 3-(4-amino-1-oxo-1,3-dihydro-isoindol-2-yl)-piperidine-2,6-dione is administered in an amount of about 1, 5, 10, or 25 mg/d, preferably in an amount of about 10 mg/d for three to four weeks, followed by one week or two weeks of rest in a four or six week cycle.

In one embodiment, an immunomodulatory compound provided herein and a second active ingredient are administered orally, with administration of an immunomodulatory compound occurring 30 to 60 minutes prior to a second active ingredient, during a cycle of four to six weeks. In another embodiment, the combination of an immunomodulatory compound provided herein and a second active ingredient is administered by intravenous infusion over about 90 minutes every cycle. In one embodiment, one cycle comprises the administration of from about 1 to about 25 mg/day of 3-(4-amino-1-oxo-1,3-dihydro-isoindol-2-yl)-piperidine-2,6-dione and from about 50 to about 200 mg/m²/day of a second active ingredient daily for three to four weeks and then one or two weeks of rest. In another embodiment, each cycle comprises the administration of from about 5 to about 10 mg/day of 4-(amino)-2-(2,6-dioxo(3-piperidyl))-isoindoline-1,3-dione and from about 50 to about 200 mg/m²/day of a second active ingredient for 3 to 4 weeks followed by one or two weeks of rest. Typically, the number of cycles during which the combinatorial treatment is administered to a patient will be from about one to about 24 cycles, from about two to about 16 cycles, and from about four to about three cycles.

### 5.6 PHARMACEUTICAL COMPOSITIONS AND DOSAGE FORMS

Pharmaceutical compositions can be used in the preparation of individual, single unit dosage forms. Pharmaceutical compositions and dosage forms provided herein comprise an immunomodulatory compound provided herein, or a pharmaceutically acceptable salt, solvate (e.g., hydrate), stereoisomer, clathrate, or prodrug thereof. Pharmaceutical compositions and dosage forms provided herein can further comprise one or more excipients.

Pharmaceutical compositions and dosage forms provided herein can also comprise one or more additional active ingredients. Consequently, pharmaceutical compositions and dosage forms herein comprise the active ingredients disclosed herein (e.g., an immunomodulatory compound and a second active agent). Examples of optional second, or additional, active ingredients are described herein elsewhere.

Single unit dosage forms provided herein are suitable for oral, mucosal, parenteral (e.g., subcutaneous, intravenous, bolus injection, intramuscular, or intraarterial), topical, transdermal or transcutaneous administration to a patient. Examples of dosage forms include, but are not limited to: tablets; caplets; capsules, such as soft elastic gelatin capsules; cachets; troches; lozenges; dispersions; suppositories; powders; gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms provided herein will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed herein will vary from one another will be readily apparent to those skilled in the art. See, e.g., Remington's Pharmaceutical Sciences, 20th ed., Mack Publishing, Easton Pa. (2000).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, the pharmaceutical compositions and dosage forms that contain little, if any, lactose other mono- or di-saccharides are provided in certain embodiments. As used herein, the term “lactose-free” means that the amount of lactose present, if any, is insufficient to substantially increase the degradation rate of an active ingredient.

Lactose-free compositions can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. In one embodiment, lactose-free dosage forms comprise active ingredients, microcrystalline cellulose, pre-gelatinized starch, and magnesium stearate.

Further encompassed are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability. Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are, for example, packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. In certain embodiment, the dosage forms comprise an immunomodulatory compound provided herein or a pharmaceutically acceptable salt, solvate (e.g., hydrate), stereoisomer, clathrate, or prodrug thereof in an amount of from about 0.10 to about 150 mg. In other embodiments, the dosage forms comprise an immunomodulatory compound provided herein or a pharmaceutically acceptable salt, solvate (e.g., hydrate), stereoisomer, clathrate, or prodrug thereof in an amount of about 0.1, 1, 2.5, 5, 7.5, 10, 12.5, 15, 17.5, 20, 25, 50, 100, 150 or 200 mg. In one embodiment, the dosage form comprises 3-(4-amino-1-oxo-1,3-dihydro-isoindol-2-yl)-piperidine-2,6-dione in an amount of about 1, 2.5, 5, 10, 15, 20, 25 or 50 mg. In certain embodiments, dosage forms comprise the second active ingredient in an amount of 1 to about 1000 mg, from about 5 to about 500 mg, from about 10 to about 350 mg, or from about 50 to about 200 mg. The specific amount of the second agent will depend on the specific agent used, the type of disease being treated or managed, and the amount(s) of an immunomodulatory compound provided herein and any optional additional active agents concurrently administered to the patient.

5.6.1 Oral Dosage Forms

Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 20th ed., Mack Publishing, Easton Pa. (2000).

In one embodiment, the dosage form is a capsule or tablet comprising 3-(4-amino-1-oxo-1,3-dihydro-isoindol-2-yl)-piperidine-2,6-dione in an amount of about 1, 2.5, 5, 10, 15, 20, 25 or 50 mg. In one embodiment, the capsule or tablet dosage form comprises 3-(4-amino-1-oxo-1,3-dihydro-isoindol-2-yl)-piperidine-2,6-dione in an amount of about 5 or 10 mg.

In certain embodiment, the oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, PA), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is typically present in from about 50 to about 99 weight percent of the pharmaceutical composition or dosage form.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation, and is readily discernible to those of ordinary skill in the art. Typical pharmaceutical compositions comprise from about 0.5 to about 15 weight percent of disintegrant or from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

In certain embodiments, the solid oral dosage form comprises an immunomodulatory compound provided herein, anhydrous lactose, microcrystalline cellulose, polyvinylpyrrolidone, stearic acid, colloidal anhydrous silica., and gelatin.

5.6.2 Controlled Release Dosage Forms

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein. Thus provided herein are single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref Biomed. Eng. 14:201 (1987); Buchwald et al., Surgery 88:507 (1980); Saudek et al., N. Engl. J. Med. 321:574 (1989)). In another embodiment, polymeric materials can be used. In yet another embodiment, a controlled release system can be placed in a subject at an appropriate site determined by a practitioner of skill, i.e., thus requiring only a fraction of the systemic dose (see, e.g., Goodson, Medical Applications of Controlled Release, vol. 2, pp. 115-138 (1984)). Other controlled release systems are discussed in the review by Langer (Science 249:1527-1533 (1990)). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient in such parenteral compositions is highly dependent on the specific nature thereof, as well as the needs of the subject.

5.6.3 Parenteral Dosage Forms

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins (see, U.S. Pat. No. 5,134,127).

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers, excipient or diluents used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of an active ingredient is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution comprising an active ingredient is an effective mode of administration. Another embodiment is a sterile aqueous or oily solution or suspension comprising an active material injected as necessary to produce the desired pharmacological effect.

Injectables are designed for local and systemic administration. Typically a therapeutically effective dosage is formulated to contain a concentration of at least about 0.1% w/w up to about 90% w/w or more or more than 1% w/w of an active ingredient to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

5.6.4 Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving the active ingredient, or a pharmaceutically acceptable salt, solvate, hydrate or prodrug thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg or 100-500 mg) or multiple dosages of the active ingredient. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, 5-35 mg or about 9-30 mg of lyophilized powder, is added per mL of sterile water or other suitable carrier. The precise amount depends upon the compound used. Such amount can be empirically determined.

### 6. EXAMPLES

Certain embodiments provided herein are illustrated by the following non-limiting example.

6.1 Differentiation of Cord Blood (CB) CD133⁺ Progenitor Cells Toward the Endothelial Lineage

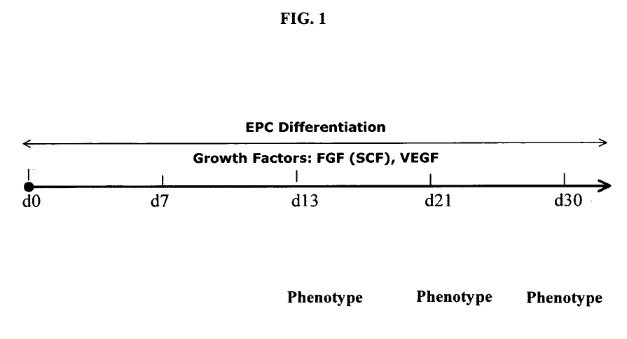

CB-CD133⁺ progenitors were obtained from AllCells and cultured in Iscove's MDM with FBS (20%) in the presence of VEGF and b-FGF or SCF for 30 to 50 days. To study the effect of immunomodulatory compounds on the generation of endothelial progenitor cells, CD133⁺ progenitor cells were cultured with or without 1-oxo-2-(2,6-dioxopiperidin-3-yl)-4-aminoisoindoline, 1,3-dioxo-2-(2,6-dioxopiperidin-3-yl)-4-aminoisoindoline, 2-amino-N-(2-(3-methyl-2,6-dioxopiperidin-3-yl)-1,3-dioxoisoindolin-4-yl)acetamide hydrochloride, or thalidomide for 30 to 50 days. Phenotypic characterization of the cells for hematopoietic and endothelial markers (CD36, CD71, Glycophorin A, and Fetal Hemoglobin) was done after 2, 3, and 4 weeks of culture using flow cytometry. Functional characterization was done after 30 days by monitoring acetylated LDL uptake in cells expressing UEA-1 lectin. Gene expression profiling was monitored at day 30 and day 40 of CD133⁺ differentiation. The procedures are illustrated in FIG. 1.

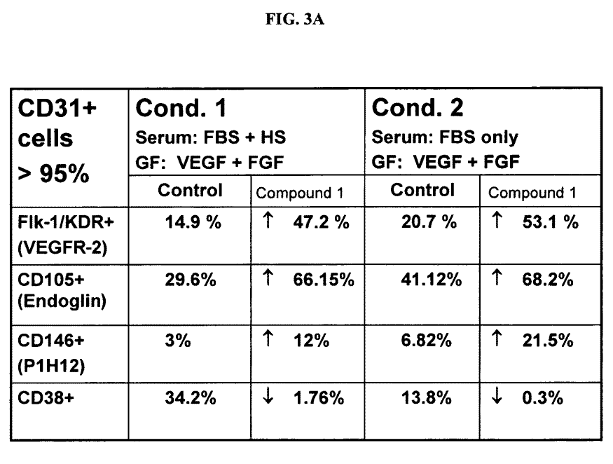

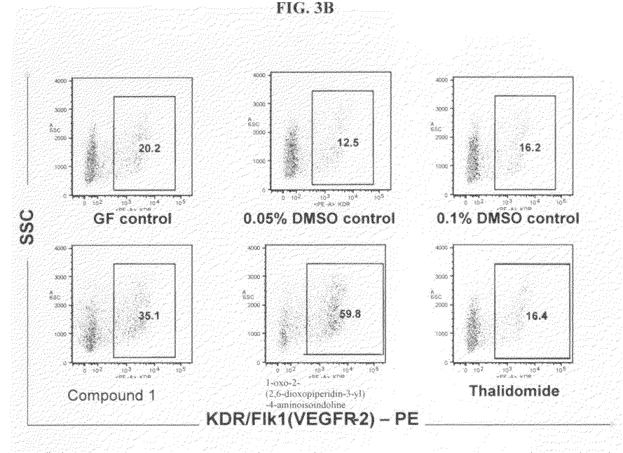

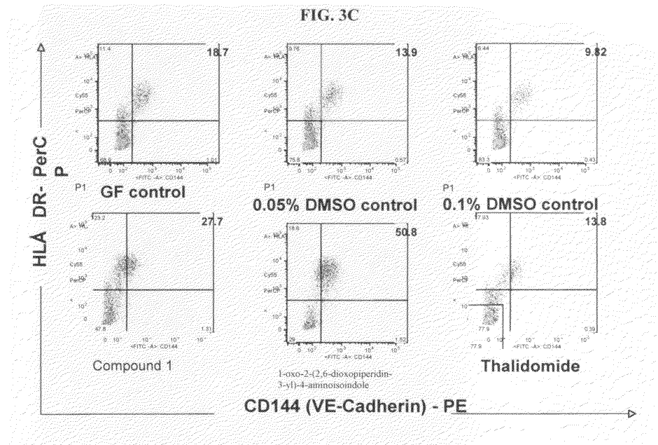

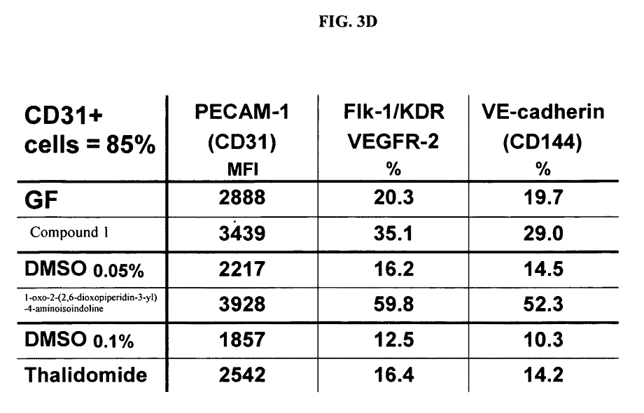

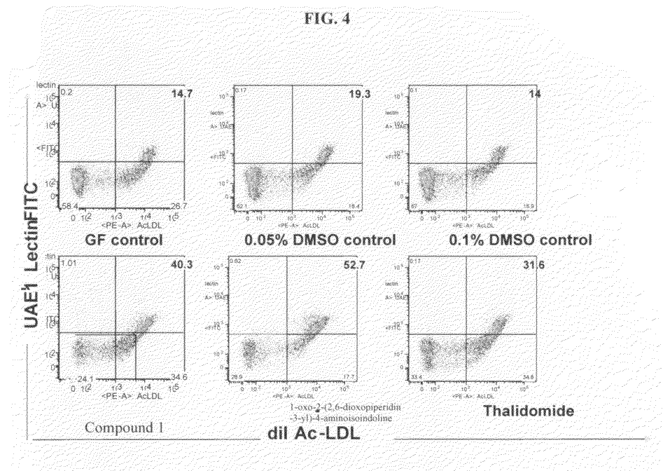

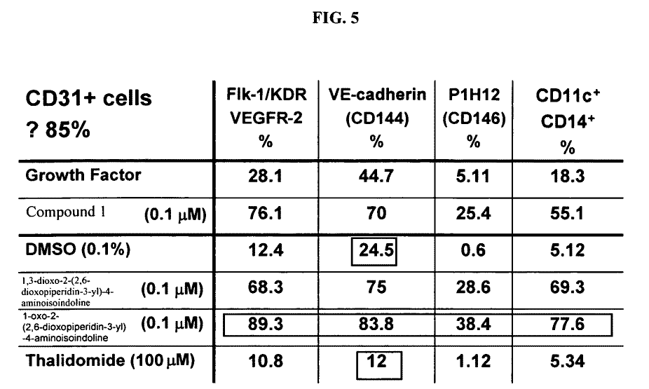

As shown in FIG. 2, CD133+ cells cultured with VEGF and B-FGF or SCF in the presence of an immunomodulatory compound exhibited a typical morphology with increased spindle shaped cells and cord-like structure formation. Further, as shown in FIGS. 3 and 5, treatment by an immunomodulatory compound increased cell surface expression of KDR (VEGFR-2) and more specific endothelial markers such as VE-cadherin (CD144), P1-H12 (CD146), and UEA 1 lection. In addition, based on the functional characterization of differentiated EPCs, which was performed by monitoring uptake of acetylated low-density lipoproteins (LDL) in cells expressing UEA-1 lectin after 30 days of culture, it was shown that treatment by an immunomodulatory compound increased the number and capacity of EPCs to take up LDL and acetylated LDL (FIG. 4).

The results indicate that an immunomodulatory compound can enhance the differentiation of EPCs.

All of the references provided herein are incorporated in their entireties by reference.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the subject matter claimed and are encompassed by the appended claims.

What is claimed is:

1. A method of treating, managing or preventing a disorder associated with endothelial dysfunction, said method comprising: 1) contacting an effective amount of an immunomodulatory compound, or a pharmaceutically acceptable salt, solvate, or stereoisomer thereof, with an endothelial progenitor cell; and 2) administering said endothelial progenitor cell contacted with an immunomodulatory compound, or a pharmaceutically acceptable salt, solvate, or stereoisomer thereof, to a patient.

2. A method of treating, managing or preventing a disorder associated with endothelial dysfunction, which comprises administering to a patient a therapeutically or prophylactically effective amount of an immunomodulatory compound, or a pharmaceutically acceptable salt, solvate, or stereoisomer thereof.

3. The method of claim 1 or 2, wherein the disorder is a cardiovascular disorder, diabetes, systemic sclerosis or chronic renal failure.

4. The method of claim 3, wherein the cardiovascular disorder is arterial hypertension, orthostatic hypotension, syncope, coronary artery disease, shock, arrhythmias, valvular heart disease, heart failure, endocarditis, pericardial disease, peripheral artery disease, a cardiac tumor, or a disease of the aorta and branches.

5. The method of claim 4, wherein the heart failure is congestive heart failure.

6. The method of claim 3, wherein the disorder is diabetes.

7. The method of claim 3, wherein the disorder is chronic renal failure.

8. The method of claim 4, which further comprises administration of a second active agent.

9. The method of claim 7, wherein the second active agent is hydrochlorothiazide, bendroflumethiazide, chlorothiazide, chlorthalidone, hydroflumethiazide, indapamide, methylclothiazide, metolazone, bumetanide, ethacrynic acid, furosemide, torsemide, amilride, spironolactone, triamterene, acebutolol, betaxolol, propranolol, metoprolol, atenolol, timolol, bisoprolol, carteolol, carvedilol, labetalol, nadolol, penbutolol, pindolol, diltiazem, verapamil, amlodipine, felodipine, isradipine, nicardipine, nifedipine, nisoldipine, benazepril, captopril, enalapril, fosinopril, lisinopril, moexipril, quinapril, ramipril, trandolapril, irbesartan, losartan, valsartan, clonidine, guanabenz, guanfacine, methyldopa, labetalol, esmolol, phentolamine, doxazosin, prazosin, terazosin, guanadrel, guanethidine, a rauwolfia alkaloid, reserpine, sodium nitro-prusside; nicardipine, fenoldopam, nitroglycerin, enalaprilat, hydlarazine, diazoxide, minoxidil, aspirin, ticlopidine, quinidine, procainamide, disopyramide, lidocaine, mexiletine, tocamide, moricizine, phenyloin, flecamide, propafenone, amiodarone, sotalol, bretylium tosylate, penicillin, amoxicillin, ampicillin, clindamycin, cephalexin, cefadroxil, azithromycin, clarithromycin, cefezolin, or a mixture thereof.

10. The method of claim 6, which further comprises administration of a second active agent.

11. The method of claim 10, wherein the second active agent is insulin, tolbutamide, chlorpropamide, acetohexamide, tolazamide, glyburide, glimepiride, metformin, acarbose, troglitazone, repaglinide, or a mixture thereof.

12. A method of promoting wound healing comprising administering to a patient a therapeutically effective amount of an immunomodulatory compound.

13. The method of claim 1, 2, or 12, wherein the immunomodulatory compound is 1-oxo-2-(2,6-dioxopiperidin-3-yl)-4-aminoisoindoline or 1,3-dioxo-2-(2,6-dioxopiperidin-3-yl)-4-aminoisoindoline.

14. The method of any of claims 1, 2, or 12, wherein the immunomodulatory compound is administered in an amount of from about 0.1 to about 150 mg per day.
